(19) United States
(12) Patent Application Publication
(10) Pub. No.: US 2010/0081568 A1
(43) Pub. Date: Apr. 1, 2010

(54) METHODS FOR PRODUCING CARBON NANOTUBES WITH CONTROLLED CHIRALITY AND DIAMETER AND PRODUCTS THEREFROM

(75) Inventor: Peter V. Bedworth, Palo Alto, CA (US)

Correspondence Address: MCDERMOTT WILL & EMERY LLP, 18191 VON KARMAN AVE., SUITE 500, IRVINE, CA, 92612-7108, US

(73) Assignee: LOCKHEED MARTIN CORPORATION, Bethesda, MD (US)

(21) Appl. No.: 12/422,918

(22) Filed: Apr. 13, 2009

Related U.S. Application Data
(60) Provisional application No. 61/046,769, filed on Apr. 21, 2008.

(51) Int. Cl. B01J 31/04; D01F 9/12; C07C 63/48; C23C 16/00

(52) U.S. Cl. 502/150; 423/447.1; 423/447.2; 562/488; 427/255.28; 977/742; 977/751; 977/842

(57) ABSTRACT

Exemplary methods of producing single-walled carbon nanotubes (SWCNTs) are disclosed. A plurality of seed cap molecules having a same diameter and a same chirality are prepared. The plurality of seed cap molecules are attached to a plurality of catalyst particles to form a plurality of catalyst-cap composites. Carbon atoms are provided to the catalyst-cap composites. Carbon nanotubes having the same diameter and the same chirality are grown on the plurality of catalyst-cap composited by exposing the composites to the carbon atoms.

### CROSS-REFERENCES TO RELATED APPLICATIONS

The present application claims the benefit of U.S. Provisional Patent Application Ser. No. 61/046,769, entitled “METHOD FOR PRODUCING CARBON NANOTUBES WITH CONTROLLED CHIRALITY AND DIAMETER,” filed on Apr. 21, 2008, which is hereby incorporated by reference in its entirety for all purposes.

### STATEMENT AS TO RIGHTS TO INVENTIONS MADE UNDER FEDERALLY SPONSORED RESEARCH OR DEVELOPMENT

Not applicable.

### BACKGROUND

1. Field

The subject technology relates generally to carbon nanotubes, and more specifically to methods for producing carbon nanotubes with controlled chirality and diameter and products produced from the methods.

2. Background

Single walled carbon nanotubes (SWCNTs) basically come in two forms: conducting and semiconducting. SWCNTs. The semiconducting form is useful for semiconductor applications (e.g., an IR detector based on photoconductivity, transistors) while the conducting form is useful for ohmic applications (e.g., radar absorbing materials, thermal materials). Currently, when commercial SWCNTs are made, a mixture of semiconducting and conducting tubes are formed. A homochiral SWCNT has a diameter specific band gap from 0 to 1.4 eV. Most applications involving SWCNTs require either conducting a mixture of semiconducting tubes or a specific bandgap tube and a mixture typically reduces the efficacy of the application.

Examples of the deleterious effect of a mixture of SWCNTs are exemplified in the fabrication of a semiconductor based device such as a carbon nanotubes field effect transistor (CNTFET), electromagnetic radiation absorbing material (EMRAM), and structural SWCNT composites. In the case of a CNTFET, a single SWCNT is to act as the semiconducting current path in a field effect transistor (FET). Hence, if the SWCNT is semiconducting, the device works. However, if the SWCNT is conducting, the device does not work, and the resulting product is defective. In the case of EMRAM, the SWCNT acts as a high aspect ratio conductive wire in an insulating matrix. Electromagnetic radiation is absorbed in this resistive (not insulating) material. The very low volume percent of SWCNTs allows highly effective graded materials to be made but the presence of semiconducting tubes requires a higher volume fraction of SWCNT to be used, thereby increasing the cost of the material and the reflectivity of the material. Currently, a structural composite material based on SWCNTs is electrically conductive. If pure semiconducting tubes were used in the composite, the structural and thermal properties would potentially stay the same but the material would be an electrical insulator. The mechanical properties depend greatly on the interaction between the SWCNT and the matrix material. The ability to choose one type of nanotube would enable optimization of the matrix/carbon nanotube (CNT) interaction. The present methods of chirality and/or diameter enhancements are practically useless for the manufacture of grown patterned structures like carbon nanotube arrays.

### SUMMARY

According to various aspects of the present disclosure, SWCNT growth methods are provided in which carbon nanotubes of a same diameter and a same chirality, hence of same conducting/semiconducting characteristics, are obtained by growing carbon nanotubes on a single type of nanotube caps having the same diameter and the same chirality. This approach provides a reliable method of producing a batch comprising either conducting or semiconducting SWCNTs with the same bandgap, and can greatly reduce defects and complexity in the manufacture of devices or products based on SWCNTs.

In one aspect of the disclosure, a method of producing single-walled carbon nanotubes is provided. The method can comprise preparing a plurality of seed cap molecules having a same diameter and a same chirality. The method can further comprise attaching the plurality of seed cap molecules to a plurality of catalyst particles to form a plurality of catalyst-cap composites. The method can further comprise providing carbon atoms. The method can further comprise growing carbon nanotubes having the same diameter and the same chirality on the plurality of catalyst-cap composites by exposing the composites to the carbon atoms.

In a further aspect of the disclosure, a single-walled carbon nanotube (SWCNT) product is provided. The SWCNT product can comprise a plurality of catalyst-cap composites. The plurality of catalyst-cap composites can comprise a plurality of catalyst particles; and a plurality of seed cap molecules having a same diameter and a same chirality and attached to the plurality of catalyst particles. The SWCNT product can further comprise a plurality of carbon nanotubes on the plurality of catalyst-cap composites. The plurality of carbon nanotubes can have the same diameter and the same chirality.

In yet a further aspect of the disclosure, a method of producing either conducting or semiconducting single-walled carbon nanotubes is provided. The method can comprise growing a plurality of nanotubes having a same diameter and a same chirality on a plurality of nanotube caps having a same diameter and a same chirality. The diameter and the chirality of the nanotubes are determined at least in part by the diameter and the chirality of the underlying nanotube caps.

In yet a further aspect of the disclosure, a single-walled carbon nanotube (SWCNT) product comprising either conducting or semiconducting carbon nanotubes is provided. The product can comprise a plurality of nanotube caps having a same diameter and a same chirality. The product can further comprise a plurality of nanotubes having a same diameter and a same chirality on the plurality of nanotube caps. The diameter and the chirality of the nanotubes are determined at least in part by the diameter and the chirality of the underlying nanotube caps.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

FIG. 1 is a flowchart illustrating an exemplary process for a controlled growth of SWCNTs having a same diameter and a same chirality according to one aspect of the subject technology.

FIG. 2 is a diagram illustrating an exemplary synthetic path for synthesizing hexacarboxycircumtrindene molecules and a time line for the synthesis according to one aspect of the subject technology.

FIG. 3A is a perspective view of a 3-D model of an exemplary catalyst-cap composite comprising a catalyst particle and a nanotube cap attached to the catalyst particle according to one aspect of the subject technology.

FIG. 3B is a diagram illustrating a (5, 5) cap that can be employed as caps according to one aspect of the subject technology.

FIG. 3C is a diagram illustrating a set of cap structures that give rise to a (10, 0) tube that can be employed as caps according to one aspect of the subject technology.

FIG. 4A is a diagram illustrating a substrate impregnated with catalyst-cap composites such as the one shown in FIG. 3A according to one aspect of the subject technology.

FIG. 4B is a diagram illustrating carbon nanotubes grown on catalyst-cap composite according to one aspect of the subject technology.

FIG. 5 is a schematic block diagram illustrating an example of a chemical vapor deposition (CVD) reactor that can be employed to grow SWCNTs according to one aspect of the subject technology.

FIG. 6 is a diagram illustrating growth of a single SWCNT column on a single seed cap according to one aspect of the subject technology.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

As discussed above, for many SWCNT application, it is desirable to grow either conducting or semiconducting SWCNTs. If the SWCNTs are semiconducting, it is also desirable to control its bandgap in order to tailor the nanotubes for a particular semiconducting application. This requires a control of its diameter and its chirality during the growth of the SWCNTs. Various embodiments of the present disclosure address and solve problems associated with conventional SWCNT growth methods. The embodiments of the present disclosure provide methods of producing SWCNTs having a same diameter and a same chirality. By providing a uniformity in the diameter and chirality, the methods lead to production of either conducting or semiconducting SWCNTs, not a mixture of both.

A great deal of effort has been applied toward the theory of carbon nanotube (CNT) growth. Currently the more well accepted mechanism can be broken into four parts: 1) a hydrocarbon is cracked to provide carbon; 2) the carbon is sorbed by a catalyst particle, 3) the CNT nucleates by forming a distribution of caps defined by catalyst and growth conditions; and 4) the tube grows by adding carbon atoms to the open end. Temperature, concentration of carbon near the tube, chemical constitution of the catalyst and size and shape of the catalyst are all important parameters in the growth process. Among the parts or steps 1)-4), the nucleation step 3) is identified as the slowest step in the reaction. In contrast to the currently-known methods, the subject technology involves initiating growth of SWCNTs on pure seed cap molecules, each acting as a nanotube cap having a same diameter and a same chirality, rather than on a distribution of caps having varying diameters and/or chiralities.

As used herein, the term “chirality” refers to molecules that are not superposable on their mirror image. Two mirror images of a molecule that cannot be superposed onto each other are referred to as enantiomers or optical isomers. As applied to nanotubes, a chiral nanotube can be specified by its chiral vector (m, n), and a particular nanotube enantiomer having (m, n) chiral vector will be hereinafter referred as (m, n) nanotube.

In one aspect of the disclosure, the term a “same diameter” may refer to a range of diameters where the range is small. For example, if seed cap molecules have a same diameter, then each (or at least 99.9% of) of the seed cap molecules has a diameter within +/−0.1% of the average diameter of the seed cap molecules. The subject technology, however, is not limited to these examples, and in another aspect, other ranges may be applicable.

In one aspect, if carbon nanotubes have a same diameter, then each (or at least 90% of) of the carbon nanotubes has a diameter within +/−0.1% of the average diameter of the seed cap molecules. The subject technology, however, is not limited to these examples, and in another aspect, other ranges may be applicable.

In one aspect of the disclosure, the term a “same chirality” may refer to having the same (m, n).

In one aspect of the disclosure, an average diameter of seed cap molecules may be, for example, any number less than 900 nm but greater than 0 nm (e.g., 800 nm, 700 nm, 500 nm, 300 nm, 200 nm, 100 nm, 50 nm, 30 nm, 25 nm, 20 nm, 10 nm, 5 nm, 3 nm, 2 nm, 1 nm).

In one aspect, an average diameter of carbon nanotubes may be, for example, any number less than 900 nm but greater than 0 nm (e.g., 800 nm, 700 nm, 500 nm, 300 nm, 200 nm, 100 nm, 50 nm, 30 nm, 25 nm, 20 nm, 10 nm, 5 nm, 3 nm, 2 nm, 1 nm).

According to one aspect of the disclosure, the term “diameter” of a structure does not imply that the structure needs to be spherical. In some embodiments, it is advantageous to have a structure that is an open geodesic poly arene molecule or an open geodesic poly hetero arene. In one aspect, the structure may contain atoms other than carbon. In one aspect, the term “diameter” may refer to a dimension of a cross section. In one aspect, the term “diameter” may refer to a dimension of the largest cross section of the structure.

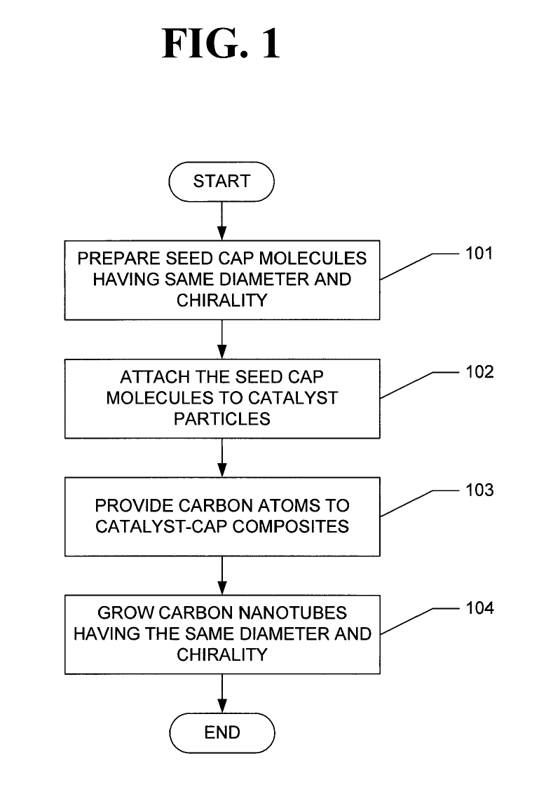

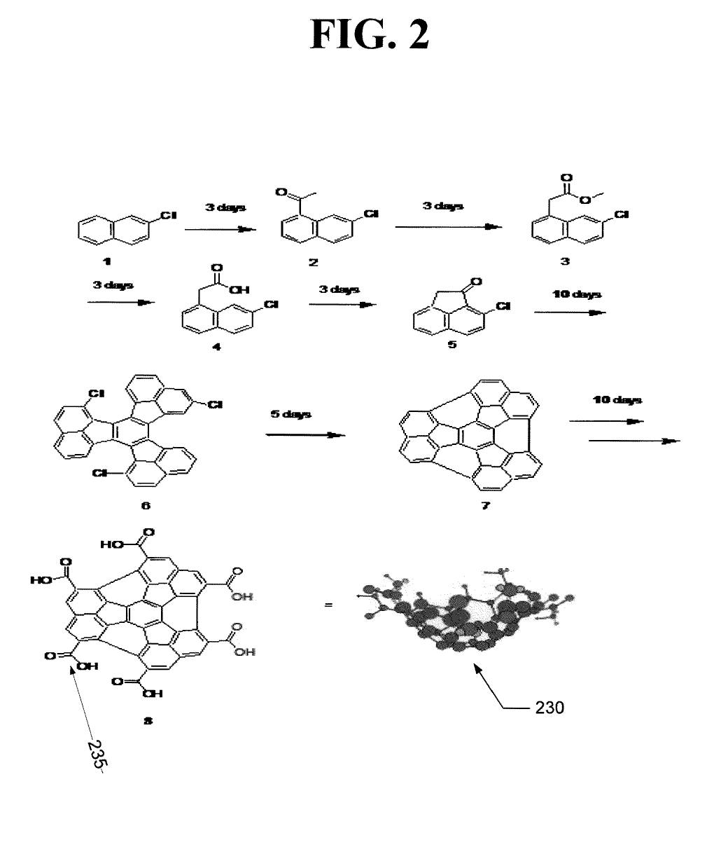

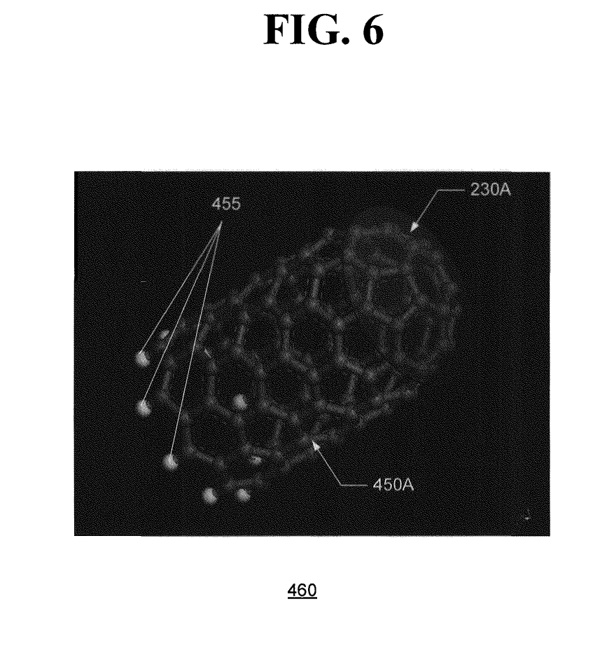

The structure of the nanotube cap can define both chirality and diameter of the SWCNT. In one aspect, SWCNT growth methods of the present disclosure are directed to preparing seed cap molecules having a same diameter and a same chirality in multi-gram quantities. FIG. 1 is a flowchart illustrating an exemplary process 100 for a controlled growth of SWCNTs having a same diameter and a same chirality according to one aspect of the subject technology. The process 100 begins at a state 101, in which a plurality of seed cap molecules having a same diameter and a same chirality are prepared. Such seed cap molecules are to be used as nanotube caps to initiate a controlled growth of SWCNTs having the same diameter and the same chirality as the seed cap molecules. The preparation of the seed cap molecules can be accomplished via traditional organic chemistry using established procedures. In one embodiment, the seed cap molecules comprise hexacarboxycircumtrindene molecules. FIG. 2 is a diagram illustrating an exemplary synthetic path in a reaction for synthesizing hexacarboxycircumtrindene molecules 230 and a time line for the synthesis according to one aspect of the subject technology. In the illustrated example, hexacarboxycircumstrindene is synthesized by functionalizing of a circumtrindene core (7) with a carboxylic acid group 235. This molecule acting as a nanotube cap gives rise to a (9,0) carbon nanotube such the one illustrated in FIG. 6 and to be described below. In one aspect of the disclosure, more than 0.01 gram of seed cap molecules can be obtained from a single reaction in a solution. In another aspect of the disclosure, more than 1 gram of seed cap molecules may be obtained from a single reaction in a solution.

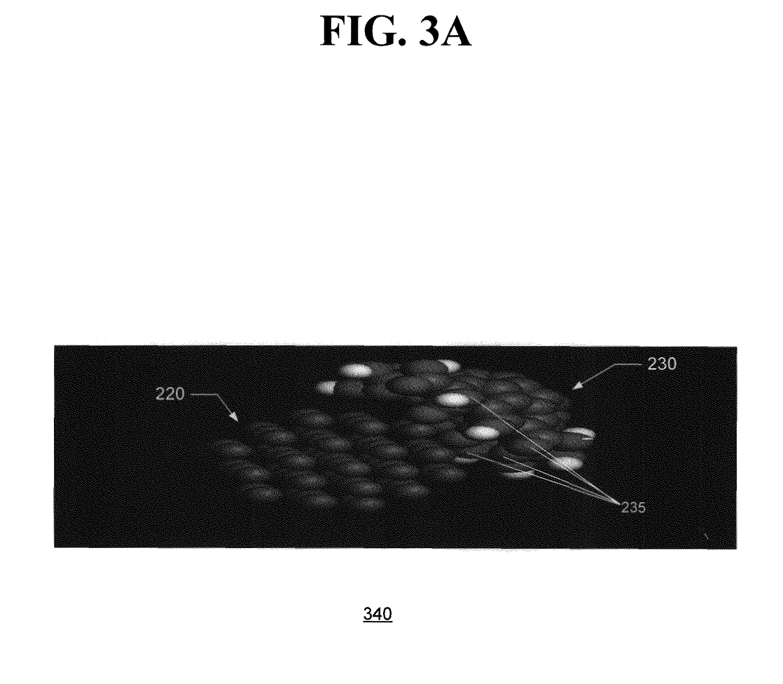

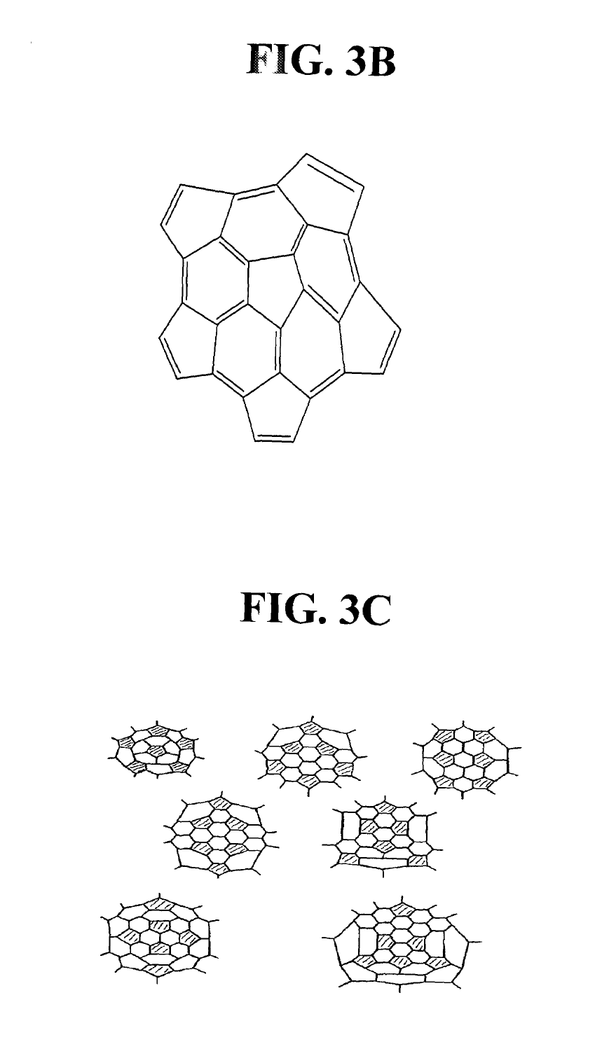

The process 100 proceeds to a state 102, in which the seed cap molecules (e.g., the hexacarboxycircumtrindene molecules 230 of FIG. 2) are attached to catalyst particles to produce a plurality of catalyst-cap composites, an example of which is shown in FIG. 3A. In the illustrated example of FIG. 3A, a catalyst-cap composite 340 comprises a hexacarboxycircumtrindene molecule 230 attached or docked on a 0.5 nm iron (Fe) cluster 220, the Fe cluster being the catalyst particle. As shown in FIG. 3A, at least three carboxylic acid groups 235 acting as attachment points are more or less equally distributed around the periphery of the seed cap molecule 230 and allow the seed cap molecule 230 to sit on the catalyst particle 220 in an optimized orientation. This optimized orientation is the same among different catalyst-cap to promote SWCNTs grown thereon to have the same diameter/chirality combination. In this sense, the carboxylic acid groups 235 of the hexacarboxycircumtrindene molecules 230 facilitate the attachment of the seed cap molecules to the catalyst particles (e.g., Fe cluster 220 of FIG. 3A).

The attachment of the seed cap molecules 230 to the catalyst particle 220 can be achieved in a number of ways. Non-limiting examples of the cap-catalyst attachment methods are now described. In one example, the nanotube cap is spin coated onto a ferrofluid catalyst. The cap complexes with the structurally defined ferric nanoparticle and causes nucleation and growth of a batch of same tubes in “forest” configuration. In another example, the nanotube cap is introduced to the ferrofluid catalyst via a dip coating process. The cap complexes with the structurally defined ferric nanoparticle and causes nucleation and growth of a batch of same tubes in “forest” configuration. In yet another example, the nanotube cap is introduced to a nano island catalyst via a dip coating. As used herein, in one aspect of the disclosure the “nano island catalyst” refers to a sub nm thick deposition of catalyst that is annealed to form a nm diameter island of catalyst. The cap complexes with the structurally defined ferric nanoparticle and causes nucleation and growth of a batch of same tubes in “forest” configuration. The attachment of the catalyst and seed can also be in solution to form a catalyst for bulk nanotube synthesis.

Before or during the cap-catalyst attachment of the state 102, native oxide coating can be formed on the exterior surface of the catalyst particles. The formation of the oxide coating naturally but an oxide is not required to form an attachment. The seed cap molecule is attached to the catalyst particle via a covalent bonding formed between the carboxylic group of the seed cap molecule and the native oxide of the catalyst particle.

The use of the ferric catalysts in the above description is for illustration only, and other types of catalysts may be employed without departing from the scope of the present disclosure. For example, an aggregate of rhenium nanoparticles may be employed as alternative catalyst particles. In some applications, the use of non ferric catalysts can be desirable in view of the fact that the carbon is relatively soluble in the ferric catalysts. Catalyst nanoparticles can include, but are not limited to, homogenous metals and alloys containing Ni, Pt, Re and Si are also catalyst candidates. Similarly, the use of the hexacarboxycircumtrindene molecule as the nanotube cap in the above description is for illustration only, and other types of caps may be employed without departing from the scope of the present disclosure. Non-limiting examples of other types of caps that may be employed include (5, 5) cap shown in FIG. 3B and a set of cap structures that give rise to a (10, 0) tube, examples of which are shown in FIG. 3C.

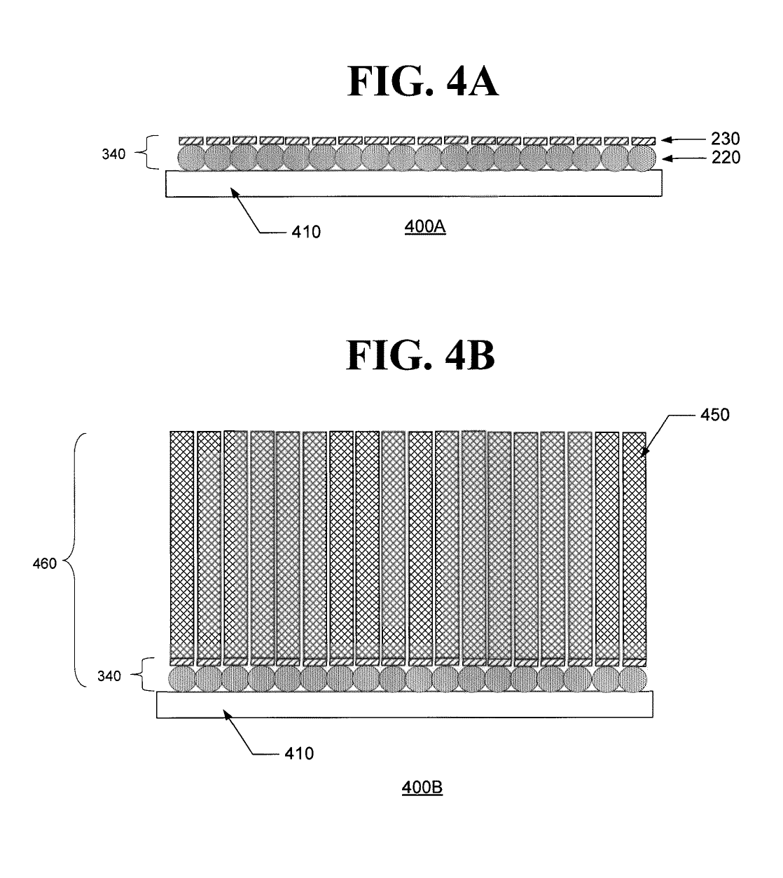

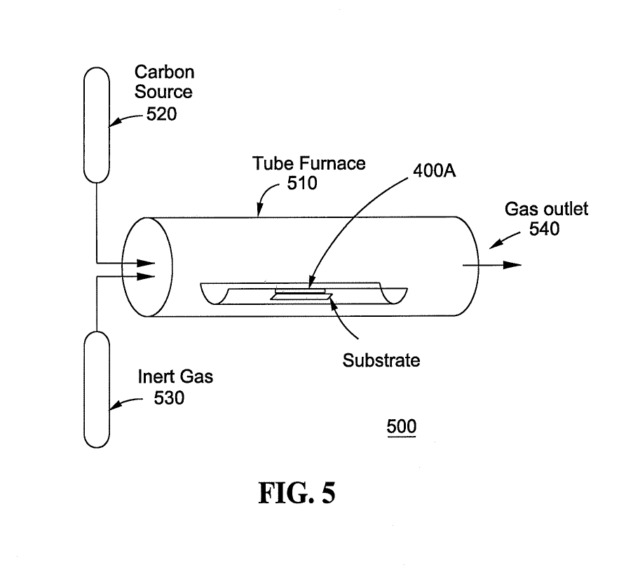

The process 100 proceeds to a state 103, in which carbon atoms are provided to the catalyst-cap composites. This provision of carbon atoms can be achieved by placing a substrate 410 impregnated (e.g., coated, deposited, or sprayed) with catalyst-cap composites such as the composite 340 illustrated in FIG. 4A. The cap/catalyst particle adduct would be formed in solution prior to impregnating the substrate, or alternatively, the substrate may be serially treated with a cap solution or vapor and a catalyst solution or vapor. The cap can also be covalently attached to the substrate. The composite impregnated substrate 400A can be placed in a chemical vapor deposition (CVD) reactor such as a CNT CVD reactor 500 shown in FIG. 5. In the illustrated example, the reactor 500 includes a tube furnace 510, a carbon source 520, an inert gas source 530, and a gas outlet 540. The composite impregnated substrate 400A is placed inside the tube furnace 510 as shown in FIG. 5, and carbon atoms from the carbon source 520 and inert gas (e.g., Ar or He) molecules from the inert gas source 530 are introduced inside the furnace 510. In particular, the gaseous mixture of the carbon atoms and the inert gas molecules flows above the composite impregnated substrate 400A. Some carbon atoms in the gaseous mixture are captured by the cap portion 230 of the catalyst-cap composites 340 (FIG. 4A) to initiate growth of SWCNTs on the cap portion. The inert gas and used carbon atoms exit the tube furnace 510 via the gas outlet 540. Examples of procedures and mechanisms for catalyzed growth of carbon nanotubes are provided in International Publication Number WO 2005/065100 and also in Ritschel et al., J. Phys. Chem. C 2007, 111, 8414-8417, both of which references are incorporated herein by reference in their entireties.

One exemplary method of growing carbon nanotubes is described below. A Re-supported MgO catalyst is prepared by wet mechanical mixing followed by a gas-producing combustion reaction with citric acid as a foaming and combustion additive. Ammoniumperrhenate (NH₄ReO₄) and magnesium nitrate ((Mg(NO₃)₂×6H₂O), which are mixed in different molar ratios (between 1 and 10), are dissolved in deionized water that contains a small amount of citric acid.

This mixture on a substrate is transferred directly into the preheated zone of a chemical vapor deposition (CVD) furnace (560° C.) where it is ignited and spontaneously burned. The reaction is accompanied by a large and strong release of different gases, which vary with the citric acid content. The total combustion process is finished after 10 minutes of exposure to result in a uniform, foamy material with a relatively high specific surface area (about 80 m²/g).

The synthesis of carbon nanotubes is carried out in a fixed-bed reactor consisting of a furnace with a quartz tube inside (diameter 40 mm). For the synthesis, a quartz boat containing the prepared catalyst material is placed in the hot zone of the horizontal reactor tube. At first, the reactor is exposed to a flow of Ar (about 250 standard cubic centimeters per minute (scc/min)) to remove the oxidizing atmosphere; afterward, the catalyst reduction is performed at 650° C. for 30 minutes in a hydrogen medium (about 150 scc/min). The temperature is increased by 6° C./min, up to the desired growth temperature between 950 and 1100° C., during the injection of CH₄ into the reactor. The CH₄ flow is stopped after the temperature has been maintained for 10 minutes. Finally, the furnace is cooled to 300° C. in a flow of hydrogen, and further cooling to room temperature is done under a flow of Ar.

For the purification, the as-grown products are sonicated in nitric acid (HNO₃) for 2 hours at room temperature. They are then filtered and washed with deionized water and dried at 110° C. for several hours.

Another exemplary method of growing carbon nanotubes is described below. Single-walled nanotubes (SWNT) by Co disproportionation can be produced by utilizing a catalyst with a Co:Re molar ratio of 1:4 under different conditions. For the SWNT production on the Co—Re/SiO₂ catalysts, 0.5 g of a calcined sample is placed in a horizontal tubular packed-bed reactor. The reactor is 12 inches long and has a diameter of 0.5 inches. After loading the catalyst, the reactor is heated in 100 scc/min H₂ flow to different temperatures in the range 600° C.-900° C. at 10° C./min.

Then, under 100 scc/min flow of He, it is heated up at the same rate to the specified reaction temperature, which ranges from 750° C. to 950° C. Subsequently, Co is introduced at a flow rate of 850 cm³/min at 84 psia for 2 hours. At the end of each run, the system is cooled down under He flow. The total amount of deposited carbon is determined by temperature-programmed oxidation. Other carbon-containing gases or fluids can be used in substitute of Co.

Returning back to FIG. 1, the process 100 proceeds to a state 104, in which a SWCNT product 460 comprising columns of SWCNTs 450 grown on the catalyst-cap composites 340 as illustrated in FIG. 4B. The SWCNT growth is achieved by initially exposing the catalyst-cap composites 340 to the carbon atoms as described above in the state 103 and then subsequently exposing the growing columns of SWCNTs 450 to the carbon atoms. FIG. 6 is a diagram illustrating growth of a single SWCNT column 450A on a single seed cap 230A. As shown in FIG. 6, during the growth, new carbon atoms 455 are added at the end of the SWCNT column 450A, and the SWCNT column 450A thus grown has the same diameter as the diameter of the underlying seed cap 230A. The SWCNT column 450A also has the same chirality as the underlying nanotube cap 230A. The result is the SWCNT product 460 (FIG. 4B) comprising a plurality of catalyst-cap composites 340, the cap composites 340 comprising a plurality of catalyst particles 220 and a plurality of seed cap molecules 230 having a same diameter and a same chirality and attached to the plurality of catalyst particles 220, and further comprising a plurality of single-walled carbon nanotubes (SWCNTs) 450 on the plurality of catalyst-cap composites, the plurality of SWCNTs 450 having the same diameter and the same chirality. Because conducting/semiconducting characteristics of the SWCNT are determined by the diameter and the chirality, the SWCNTs 450 having the same diameter and the same chirality are either conducting or semiconducting with the same electrical properties (e.g., the same conductivity if conducting or the same bandgap if semiconducting).

In the exemplary embodiments described above, the diameter and the chirality of the carbon nanotubes are respectively the same as the diameter and the chirality of the nanotube caps that the nanotubes are grown on. Alternatively, however, either the diameter or the chirality or both may be different between the nanotubes and the caps. For example, depending on the particular seed cap molecule employed, the diameter of the carbon nanotube may be different (e.g., slightly larger or smaller) than the diameter of the cap. Similarly, the chirality of the carbon nanotube may be different from the chirality of the cap. However, the grown nanotubes of the SWCNT product can still be of the same diameter and the same chirality as these parameters of the nanotubes are nevertheless determined by the corresponding parameters of the underlying caps.

After or during the growth process, the SWCNTs can be subjected to a physical analysis to determine some relevant properties. The analysis of the SWCNTs can be carried out using a Raman spectrometer and/or a photoluminescence and photoluminescence excitation spectrometer. For example, in each part of a Raman spectrum, the radial breathing mode (RBM), the disorder induced mode (D mode), and the high-energy mode (HEM) can be used to access different properties of single-walled carbon nanotubes. Out of all Raman modes the radial breathing mode is unique to SWCNTs. In the high energy range around 1600 cmⁱ a SWCNT can show a characteristic double-peak structure. Non-limiting examples of properties of the SWCNTs that can be determined from a nanotube Raman spectrum include:
1) Presence A Raman spectrum showing the radial breathing mode and the double-peak structure of the high-energy modes can prove the presence of single-walled carbon nanotubes in a sample or the presence of an isolated nanotube in the laser focus.
2) Orientation The orientation of the tubes can be measured by the intensity of the Raman spectrum. The signal is always strongest when the laser light is polarised along the nanotube axis.
3) Diameter The frequency of the radial breathing mode is proportional to the inverse of the nanotube diameter. The diameter of carbon nanotubes can be estimated by measuring the RBM frequency. Out of all possible methods to find the diameter of a tube Raman scattering is by far the easiest and fastest method.
4) Conducting/semiconducting Raman scattering can distinguish between metallic and semiconducting nanotubes. In metallic carbon nanotubes the lower high-energy mode is strongly broadened and shifted to smaller energies (1540 cm⁻¹). This so-called metallic spectrum appears only in metallic tubes and for a properly chosen energy of the incoming laser light.
5) Energies A resonance profile, i.e., the dependence of the Raman intensity on laser energy, yields the energies of the excited electronic states or the optical transition energies. Particularly suited for these studies is the radial breathing mode, because the resonance window is narrow. Combined with photoluminescence and photoluminescence excitation spectroscopy the Raman spectrum allows for identification of particular (n₁,n₂) carbon nanotubes.
6) Strain The phonon frequencies shift when the bond lengths or angles change. For example, a contraction of a tube along its axis or a change in the nanotube diameter change the carbon-carbon distance. The dependence of the phonon frequencies on the strain can be used to study the mechanical properties of the tubes or their response to electrochemical doping.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A method of producing single-walled carbon nanotubes, the method comprising:
preparing a plurality of seed cap molecules having a same diameter and a same chirality;
attaching the plurality of seed cap molecules to a plurality of catalyst particles to form a plurality of catalyst-cap composites;
providing carbon atoms; and
growing carbon nanotubes having the same diameter and the same chirality on the plurality of catalyst-cap composites by exposing the composites to the carbon atoms.

2. The method of claim 1, wherein each of the plurality of seed cap molecules comprises a hexacarboxycircumstrindene.

3. The method of claim 2 further comprising functionalizing a circumtrindene core with a carboxylic acid group to obtain the hexacarboxycircumstrindene.

4. The method of claim 3, wherein the carboxylic acid group is used to attach a seed cap molecule to a catalyst particle.

5. The method of claim 4 further comprising forming native oxide coatings on plurality of catalyst particles.

6. The method of claim 5 further comprising attaching each of the plurality of seed cap molecules to each of the plurality of catalyst particles via a covalent bonding formed between the carboxylic group of the seed cap molecule and the native oxide of the catalyst particle.

7. The method of claim 1, wherein each of the plurality of catalyst particles comprises a ferric catalyst.

8. The method of claim 1, wherein each of the plurality of catalyst particles comprises a rhenium nanoparticle.

9. The method of claim 1 further comprising subjecting a substrate comprising the plurality of catalyst-cap composites to a chemical vapor deposition process.

10. A single-walled carbon nanotube (SWCNT) product, the SWCNT product comprising:
a plurality of catalyst-cap composites comprising:
a plurality of catalyst particles; and
a plurality of seed cap molecules having a same diameter and a same chirality and attached to the plurality of catalyst particles, and
a plurality of carbon nanotubes on the plurality of catalyst-cap composites, the plurality of carbon nanotubes having the same diameter and the same chirality.

11. The SWCNT product of claim 10 further comprising a substrate, the plurality of catalyst-cap composites disposed above the substrate and affixed to the substrate.

12. The SWCNT product of claim 10, wherein each of the plurality of seed cap molecules comprises hexacarboxycircumstrindene.

13. The SWCNT product of claim 10, wherein each of the plurality of catalyst particles comprises a ferric catalyst.

14. The SWCNT product of claim 10, wherein each of the plurality of catalyst particles comprises a rhenium nanoparticle.

15. The SWCNT product of claim 10, wherein the plurality of carbon nanotubes comprise (9,0) nanotubes.

16. A single-walled carbon nanotube (SWCNT) product comprising either conducting or semiconducting carbon nanotubes, the product comprising:
a plurality of nanotube caps having a same diameter and a same chirality; and
a plurality of nanotubes having a same diameter and a same chirality on the plurality of nanotube caps,
wherein the diameter and the chirality of the nanotubes are determined at least in part by the diameter and the chirality of the underlying nanotube caps.

17. The product of claim 16, wherein the diameter and the chirality of the plurality of the nanotubes are respectively the same as the diameter and the chirality of the plurality of the nanotube caps.

18. The product of claim 17, wherein the plurality of seed cap molecules are obtained via a reaction in a solution, wherein more than 0.01 grams of the plurality of seed cap molecules can be obtained from reaction in the solution.
